(19) United States
(12) Patent Application Publication
(10) Pub. No.: US 2022/0387569 A1
(43) Pub. Date: Dec. 8, 2022

(54) INDUCED PLURIPOTENT STEM CELL-BASED CANCER VACCINES

(71) Applicant: Khloris Biosciences, Inc., Mountain View, CA (US)

(72) Inventors: Stephen D. Wolpe, Boyds, MD (US); Lynne A. Bui, Saratoga, CA (US); Nigel G. Kooreman, Den Haag (NL)

(73) Assignee: Khloris Biosciences, Inc., Mountain View, CA (US)

(21) Appl. No.: 17/827,957

(22) Filed: May 30, 2022

Related U.S. Application Data
(60) Provisional application No. 63/195,609, filed on Jun. 1, 2021.
(60) Provisional application No. 63/233,141, filed on Aug. 13, 2021.

(51) Int. Cl. A61K 39/00; A61P 37/04

(52) U.S. Cl. CPC A61K 39/0011; A61P 37/04; A61K 2039/5154; A61K 2039/5158; A61K 2039/55561

(57) ABSTRACT

In one embodiment, the present application discloses a mammalian autologous vaccine or allogeneic vaccine comprising an effective amount of a mammalian induced pluripotent stem cells (iPSCs) obtained by reprogramming of somatic cells from a patient; wherein the autologous vaccine or the allogeneic vaccine expresses a gene selected from the group consisting of ASTE1, BIRC5, CDCA1, CDKN2A, DEPDC1, EGFR, ERBB2, FOXM1, GPC3, HJURP, HSPA8, HSP90B1, IDH1, IDO1, IGF2BP3, IMPS, KIF20A, KIF20B, MELK, MGAT5, NUF2, PMEL, RAS, TAF1B, TOMM34, TTK, TP53, VEGFR1 and VEGFR2; and wherein the autologous vaccine or the allogeneic vaccine induces an immune response from the patient for the treatment of cancer.

### CROSS-REFERENCE TO RELATED APPLICATION

This application claims the benefit under 35 USC 119(e) of Application No. 63/195,609, filed Jun. 1, 2021, and of Application No. 63/233,141, filed Aug. 13, 2021, the entire contents of which are incorporated into this application by reference.

### BACKGROUND OF THE INVENTION

Yamanaka and colleagues (Yamanaka S. et al. Cell 126:663-76, 2006; reviewed in Shi Y. et al. Nat Rev Drug Discov. 16:115-130, 2017) developed methods for reverting adult cells to an embryonic state (“reprogramming”). These induced pluripotent stem cells (iPSCs) can be generated by introducing four transcription factors into adult somatic cells, which transform their transcriptional and epigenetic state to a pluripotent one that closely resembles embryonic stem cells (ESCs).

Kooreman et al. (US2019/0290697, which is hereby incorporated by reference in its entirety) noted a number of similarities in the gene expression of iPSCs and cancer cells and developed a vaccine using iPSCs that prevented the growth of a number of cancers. These authors determined that the best efficacy seen for this vaccine was to obtain cells from an individual, reprogram those cells into iPSCs and use that individual's iPSCs together with an adjuvant as a vaccine for that individual (“autologous” vaccination). These investigators also identified a particular type of adjuvant that is comprised of a short, synthetic, unmethylated CpG motif-based oligodeoxynucleotide (“CpG”) as being the most efficacious in their models.

### SUMMARY OF THE INVENTION

The present inventors discovered that there is a need to develop iPSCs that can present additional antigens to potentiate the responses to individual cancers. There also exists a need to develop off-the-shelf products where iPSCs from one individual can be used to treat genetically unrelated individuals (“allogeneic vaccination”) as well as to identify other adjuvants and targeting strategies that may result in stronger or more effective immune responses. There also exists a need to develop criteria for judging the effectiveness of an iPSC cancer vaccine during its manufacture (“release criteria”) and after being administered to an individual (“biomarkers”).

In one embodiment, the present application provides compositions and methods for the generation of a cancer vaccine targeting multiple types of cancer, either prophylactically or therapeutically. In one aspect, the vaccine comprises an adjuvant and iPSCs or mini-intronic plasmid-generated iPSCs (MIP-iPSCs). In one variation, the adjuvant is admixed with the iPSCs. The adjuvant may be loaded on to iPSCs by incubating the cells with said adjuvant. In one variation, the adjuvant is genetically encoded in the iPSCs.

In one embodiment, the cancer vaccine is autologous (or autogenous). As used herein, an autologous or autogenous vaccine refers to a vaccine that may be prepared by the reprogramming of cells from an individual and used to provide immunity to the same individual.

In one embodiment, the cancer vaccine is an off-the-shelf, allogeneic product.

In one embodiment, methods are provided for autologous and allogeneic cancer vaccine generation and vaccination regimen. The inventors have surprisingly found that, in one particular aspect, allogeneic iPSC vaccines are not as effective as autologous vaccines and have developed methods for improving the efficacy of both autologous and allogeneic vaccinations.

In one aspect, the cancer vaccine is genetically engineered to improve efficacy of antigen presentation.

In another embodiment of the compositions and methods, the autologous or allogeneic vaccine is genetically modified to express an antibody or an antibody fragment. The antibody may be a monoclonal antibody, a humanized antibody, a chimeric antibody, a single chain antibody, an antibody fragment, or combinations thereof. The antibodies may be secreted by the iPS cells, or they may be expressed at the cell surface as transmembrane protein. The antibodies may be reactive, for example, towards cancer antigens, cytokines, growth factors or their receptors, or proteins expressed on the surface of T-cells. The term “antibody” may be used to define any integral cell surface protein capable of binding a ligand including, but not limited to, an immunoglobulin comprised of a heavy and light chain; a single-chain variable fragment (scFv) that is a fusion protein of the variable regions of an immunoglobulin heavy (VH) and light chain (VL); a camelid single-domain antibody; a nanobody or other variation known in the art.

In one variation, the iPS cells are genetically modified to express anti-CD28 and anti-CD80 antibodies on their cell surface.

In one variation, the autologous or allogeneic vaccine is genetically engineered to express cell surface ligands selected from the list consisting of ICAM1, LFA-1, LFA-3, CD80, CD81, CD28, ICOS, 4-1BB, anti-DEC-205 antibody, anti-CLEC9A (DNGR) antibody, anti-DCIR-2 antibody, anti-DECTIN antibody, anti-ASGPR antibody, anti-mannose receptor antibody and anti-CLEC12 (DCAL-2) antibody. In another variation, the autologous or allogeneic vaccine is genetically engineered to express connexin-43. In another variation, the autologous or allogeneic vaccine is genetically engineered to secrete proteins selected from the group consisting of XCR1, CCL3, CCL4, CCL5, CCL20, CCL25 and FLT3L or combination thereof. In one variation, the allogeneic vaccine is genetically engineered to express CCL3 and FLT3L. In another variation, iPSCs are genetically engineered to secrete proteins selected from the group consisting of GM-CSF, INF-alpha, INF-beta, IL-2, IL-12, IL-15 and IL-21, or a combination thereof. In another variation, the allogeneic vaccine expresses IL-15 on its cell surface. In another variation, iPSCs are genetically engineered to express proteins selected from the group consisting of gp96, hsp90, hsp70, CD91, calreticulin and LOX-1. In a particular variation, the iPSCs are genetically engineered to express hsp70. In another variation, the autologous or allogeneic vaccine is genetically engineered to express a cell surface protein selected from the group consisting of B7, OX40, CD28, CD40L, TLR4, CD70, MHC Class I, MHC Class II and OX40L. In another variation, the vaccine is engineered to express OX40.

In one embodiment, the autologous or allogeneic vaccine is genetically engineered to contain an inhibitory RNA. In one variation, the inhibitory RNA is selected from the group consisting of an antisense RNA, an siRNA, an shRNA, a miRNA, a lncRNA, a pri-miRNA, an antisense oligonucleotide and a pre-miRNA. In one variation, the inhibitory RNA inhibits expression of a gene selected from the group consisting of MHC Class I, MHC Class II, Beta2 microglobulin and LAMP. In another variation, the inhibitory RNA inhibits Beta2 microglobulin. In one variation, the inhibitory RNA inhibits expression of a gene selected from the group consisting of PD-1, PDL-1, PDL-2, Nodal, cytokine signaling 1 (SOCS1), IL-10, IL-10R, TGF-β and TGF-β. In one variation, the inhibitory RNA inhibits expression of TGF-β. In one variation the inhibitory RNA is expressed at sufficiently high concentration to inhibit expression of one or more of these genes in antigen-presenting cells that take up the iPSC vaccine. It is understood that those skilled in the art can use methods other than inhibitory RNA, for example methods involving CRISPR, to inhibit the expression of particular genes in the autologous or allogeneic vaccine. See Li, H.L. et al. Methods 101:27-35, 2015; Terns M.P. Mol. Cell 72:404-412.

In another aspect, the autologous or allogeneic vaccine is administered in combination with a small molecule drug. In one embodiment, the drug is selected from the group consisting of an HDAC inhibitor, a bromodomain inhibitor, a Vps34 kinase inhibitor, a PRMT5 inhibitor, an autophagy inhibitor, an angiogenesis inhibitor, a vascular disruption agent, an activator of the STING pathway and an activator of the Toll receptor pathway. In another variation, the small molecule drug is an activator of STING plus an activator of the Toll receptor pathway.

In one variation, the HDAC inhibitor is selected from the group consisting of valproic acid, givinostat, belinostat, entinostat, mocetinostat, practinostat, chidamide, quisinostat abexinostat, vorinostat, romidepsin, panobinostat and belinostat. In another variation, the HDAC inhibitor is vorinostat.

In one variation, the BET inhibitor is selected from the group consisting of I-BET 151 (GSK1210151A), I-BET 762 (GSK525762), OTX-015, TEN-010. CPI-203 and CPI-0610. In one variation, the BET inhibitor is CPI-0610. In another variation, the Vps34 kinase inhibitor is selected from the group consisting of SB02024 and SAR405. In yet another variation, the PRMT5 inhibitor is GSK3326595.

In one variation, the autophagy inhibitor is selected from the group consisting of 3-methyladenine, bafilomycin A1, chloroquine, hydroxychloroquine, N-2˜-(1H-benzimidazol-6-yl)-N˜4˜-(5-cyclobutyl-1H-pyrazol-3-yl)quinazoline-2,4-diamine, MRT68921, MRT67307, SBI-0206965, ULK100, ULK101 and SB02024. In another variation, the autophagy inhibitor is SB02024.

In one variation, the angiogenesis inhibitor is selected from the group consisting of axitinib, bevacizumab, cabozantinib, everolimus, lenalidomide, lenvatinib mesylate, pazopanib, regorafenib, sorafenib, sunitinib, thalidomide, vandetanib and ramucirumab. In another variation, the angiogenesis inhibitor is sorafenib.

In one variation, the vasculature disrupting agent is selected from the group consisting of combretastatin, AVE8062, ZD6126, ABT-571, MN-029, CKD516, OXi8006, 5,6-dimethylxanthenone 4-acetic acid, combretastatin A-4 phosphate, ZD6126, Oxi4503, DMXAA and the dolatastatin derivative TZT-1027. In another variation, the vasculature disrupting agent is DMXAA.

In one variation, the STING activator is selected from the group consisting of ADU-S100, MK-1454, MK-2118, BMS-986301, SR-717, GSK3745417, SB-11285, IMSA-101, c-di-GMP, c-di-AMP and cGAMP. In another variation, the STING activator is SR-717.

In one variation, the Toll pathway activator is selected from the group consisting of diacyl lipopeptide, triacyl lipopeptide, flagellin, poly I:C, hexa-acetylated lipid A, monophosphoryl lipid A, gardiquimod, imiquimod and R848. In another variation, the Toll pathway activator is imiquimod.

In another variation, the autologous or allogeneic vaccine is co-administered with an antibody selected from the group consisting of anti-CD47, rituximab, cetuximab, daratumumab, trastuzumab, trastuzumab emtansine, pertuzumab, panitumumab, ramucirumab, necitumumab and blinatumomab. In another variation, the autologous or allogeneic vaccine is co-administered with blinatumomab. In another variation, the autologous or allogeneic vaccine is co-administered with a small molecule drug selected from the group consisting of ibrutinib, acalabrutinib and galuniseritib. In another variation, the autologous or allogeneic vaccine is co-administered with ibrutinib.

In another aspect, the pluripotent stem cells are induced to undergo a specific type of cell death. The inventors have surprisingly discovered that the pluripotent stem cells do not need to be viable at the time of injection in order to elicit the desired anti-cancer immunity. In one embodiment, iPSC that are killed by in vitro administration of oxaliplatin or doxorubicin and induced to express calreticulin prior to admixture with adjuvant are particularly potent.

In one variation, the cell death-inducing agent is selected from the group consisting of glutamate, sorafenib, ML162, FIN56, FIN02, erastin, sulfazine, RSL3, Ki8751, SGX-523, AZD7762, KW-2449, NVP-TAE684, AZD4547, TG-101348, bleomycin, axitinib, cytochalasin B, dasatinib, SNX-2112, Semagacestat, CHIR-99021, B02, olaparib, silmitasertib, tanespimycin, nintedanib, ML031, canertinib, SMER-3, BCL-LZH-4, SN-38, tamatinib, ML334 diastereomer, analogues, salts or derivatives thereof. In another variation, the cell death-inducing agent is bleomycin.

Preparations can be frozen and thawed prior to administration in a patient without loss of activity. In one variation, the iPSCs are admixed with an adjuvant prior to freezing. In one variation the adjuvant is selected from the group consisting of saponin formulations, virosomes, virus like particles, non-toxic derivatives of enterobacterial lipopolysaccharide (LPS), immunostimulatory oligonucleotides (e.g. an immunostimulatory oligonucleotide containing a CpG motif), mineral containing compositions, oil-emulsions, polymers, micelle-forming adjuvants (e.g., a liposome), immunostimulating complex matrices (e.g., ISCOMATRIX), particles, squalene, phosphate, cationic liposome-DNA complexes (CL DC), DDA, DNA adjuvants, gamma-insulin, ADP-ribosylating toxins, detoxified derivatives of ADP-ribosylating toxins, Freund's complete adjuvant, Freund's incomplete adjuvant, muramyl dipeptides, monophosphoryl Lipid A (MPL), poly IC, CpG oligodeoxynucleotides (ODNs), imiquimod, QS21, AS101, adjuvant system ASO, adjuvant system AS02, adjuvant system AS03, MF59®, poly di(carboxylatophenoxy)phosphazene; derivatives of lipopolysaccharides such as monophosphoryl lipid A, muramyl dipeptide (MDP; Ribi), threonyl-muramyl dipeptide (t-MDP; Ribi); OM-174; cholera toxin (CT), and Leishmania elongation factor and aluminum or aluminum salts (e.g. alum, aluminum phosphate, aluminum hydroxide). In another variation, the adjuvant is AS101.

Release assays can be used to qualify particular lots of autologous or allogeneic iPSCs as having appropriate characteristics to be used in a vaccine preparation whereas qualification assays can be used to validate a particular manufacturing process. Release or qualification assays can be based on the expression of the appropriate genes by measuring mRNA, protein or carbohydrates in the iPSCs according to methods known in the art including, but not limited to, PCR, RNAseq, flow cytometry, ELISA and immunocytochemistry. In addition to criteria previously published for iPSCs for regenerative medicine (Sullivan et al. 2018) the inventors have identified genes whose expression in iPSC make them particularly useful as vaccines for particular cancers. These genes are listed in Table 1.

TABLE 1
Genes Useful as Release or Qualification Assays
for Manufacturing of iPSC for Cancer Vaccination:

| | |
|---|---|
| Astrocytoma | IDH1 |
| Bladder | DEPDC1 KIF20B |
| Breast | BIRC5 CDCA1 DEPDC1 |
| | ERBB2 KIF20A KIF20B |
| Cervical | FOXM1 HJURP MELK |
| Colorectal | ASTE1 IGF2BP3 TAF1B |
| | TOMM34 VEGFR1 |
| | VEGFR2 |
| Esophageal | CDCA1 IGF2BP3 IMP3 |
| | TTK |
| Gastric | ERRB2 |
| Glioblastoma | EGFR HSP90B1 |
| Head and Neck | CDCA1 CDKN2A IMP3 |
| Liver | GPC3 HSPA8 |
| Melanoma | HSP90B1 MGAT5 PMEL |
| NSCLC | ERBB2 HSP90B1 IDO1 |
| | IMP3 NUF2 TTK TP53 |
| | VEGFR1 VEGFR2 |
| Ovarian | BIRC5 ERBB2 FOXM1 |
| | HJURP MELK VEGFR1 |
| | VEGFR2 |
| Pancreatic | ERBB2 HSPA8 KIF20A |
| | RAS TP53 VEGFR1 |
| | VEGFR2 |
| Prostate | BIRC5 ERBB2 |

One or more genes selected from Table 1 can be used to qualify a manufacturing lot of iPSCs for use in an autologous vaccine for a patient with a cancer listed in the Table. In another embodiment, one or more genes selected from Table 1 can be used to qualify a manufacturing lot of iPSCs.

In one embodiment, one or more genes selected from Table 1 can be used to follow the immune response of a patient to the vaccine. In one variation the antibody response to one or more genes selected from Table 1 can be followed using assays including, but not limited to, ELISA or Luminex. In one variation the cellular immune response to one or more genes selected from Table 1 can be followed using assays including, but not limited to, Elispot or cytotoxic T cell killing assays.

### DETAILED DESCRIPTION OF THE INVENTION

Compositions and methods are provided for the generation of the pluripotency vector, generation of iPSCs with this vector, establishing the cancer vaccine and vaccinating subjects prophylactically and therapeutically.

The cancer vaccine, as used herein, is the use of the host's pluripotent stem cells in combination with the adjuvant to prime the same host's immune system in targeting cancer cells.

The hosts are generally mammals, including but not limited to humans, dogs, cats, or horses. Laboratory animals, such as rodents are of interest for the cancer selections studies, epitope screening and mechanistic studies. Larger animal studies, e.g. pig and monkey are of interest for safety studies.

For the purpose of invention, pluripotent cells may be autologous, allogeneic or xenogeneic with respect to the recipient.

“Treatment” refers to both therapeutic treatment and prophylactic or preventative measures. Those in need of treatment include those already with the disorder as well as those in which the disorder is to be prevented. In another embodiment, “treating” or “treatment” of any condition or disorder refers, in certain embodiments, to ameliorating a condition or disorder that exists in a subject, including prophylactically. In another embodiment, “treating” or “treatment” includes ameliorating at least one physical parameter, which may be indiscernible by the subject. The term “treating” or “treatment” includes modulating the condition or disorder, either physically (e.g., stabilization of a discernible symptom) or physiologically (e.g., stabilization of a physical parameter) or both. The term “treating” or “treatment” includes delaying the onset of the condition or disorder. In addition, the term “treating” or “treatment” includes the reduction or elimination of either the condition (e.g., pain) or one or more symptoms (e.g., pain) of the condition (e.g., cancer), or to retard the progression of the condition or of one or more symptoms of the condition, or to reduce the severity of the condition or of one or more symptoms of the condition. In one variation, “treating” or “treatment” includes administering a vaccine described herein prophylactically.

As used herein, “mammal” refers to any animal classified as a mammal, including humans, domestic and farm animals, and zoo, sports, or pet animals, such as dogs, horses, cats, cows, etc. In one aspect, the mammal is human.

As used herein, “pluripotency” and “pluripotent” stem cells means that such cells have the ability to differentiate into all types of cells in an adult organism. The term “induced pluripotent stem cell” encompasses pluripotent cells, that, like embryonic stem cells (ESCs), can be cultured over a long period of time while maintaining the ability to differentiate into all types of cells in an organism, but that, unlike ESCs (which are derived from the inner cell mass of blastocysts), are derived from differentiated somatic cells, that is, cells that had a narrower, more defined potential and that in the absence of experimental manipulation could not give rise to all types of cells in the organism. By “having the potential to become iPSCs” it is meant that the differentiated somatic cells can be induced to become, i.e. can be reprogrammed to become, iPSCs. That is, the somatic cell can be induced to dedifferentiate so as to establish cells having the morphological characteristics, growth ability and pluripotency of pluripotent cells. iPSCs have a human ESC-like morphology, growing as flat colonies with large nucleo-cytoplasmic ratios, defined borders and prominent nucleoli. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT and zfp42. In addition, pluripotent cells are capable of forming teratomas. In addition, they are capable of forming or contributing to ectoderm, mesoderm, or endoderm tissues in a living organism.

The term “viable”, as used, for example, in the clause “ . . . pluripotent stem cells do not need to be viable . . . ” means that the cells do not need to have intact membranes or be metabolically active, as assessed by commonly used viability stains such as trypan blue, propidium iodide, 3-[4,5-dimethylthiazol-2-yl]-2,5 diphenyl tetrazolium bromide (MTT) and similar assays (reviewed in Kamiloglu et al., 2020, Food Frontiers 1:332-349.

Somatic cells, with a combination of three, four, five, six, or more factors can be de-differentiated/reprogrammed to a state apparently indistinguishable from embryonic stem cells (ESCs); these reprogrammed cells are termed “induced pluripotent stem cells” (iPSCs, iPCs, iPSCs) and can be produced from a variety of tissues (Shi Y. et al., Induced pluripotent stem cell technology: a decade of progress. Nat Rev Drug Discov. 2017 February; 16(2):115-130).

The vaccines may also comprise an adjuvant. Adjuvants useful in vaccine are well known to those of skill in the art, and accordingly, the selection of an appropriate adjuvant can be performed routinely by one of skill in the art upon review of the present application. Examples of useful adjuvant include, but are not limited to, complete and incomplete Freund's, mineral gels such as aluminum hydroxide, surface active substances such as lysolecithin, pluronic polyols, polyanions, peptides and oil emulsions. Adjuvants that are particularly useful are those that stimulate cellular immunity including, but not limited to, CG-enriched oligodeoxynucleotides (CpG), Bacillus Calmette-Guerin (BCG), activators of the cGAS-STING pathway, MPL (3-O-desacyl-4′-monophosphoryl lipid A), ASO4 adjuvant and mycobacteria cell wall peptidoglycans. In some embodiments, the vaccine is an injectable composition that is sterile, pyrogen free, formulated to be isotonic and free of particulates. The standards of purity required for injectable compositions are well known as are the production and purification methods used to prepare injectable compositions. The vaccines may be administered by any means known in the art. Pharmaceutical injectable compositions may be administered parenterally, i.e., intravenous, subcutaneous and intramuscular. In some embodiments, pharmaceutical vaccine compositions may be administered intranasally or to tissue in the oral cavity such as by administration sublingually or to buccal tissue.

The term “stem cell” refers to an unspecialized cell that is capable of replicating or self-renewing itself and developing into specialized cells of a variety of cell types. The product of a stem cell undergoing division is at least one additional cell that has the same capabilities as the original cell. Pluripotent stem cells are cells derived from any kind of tissue (usually embryonic tissue such as fetal or pre-fetal tissue), which stem cells have the characteristic of being capable under appropriate conditions of producing progeny of different cell types that are derivatives of all of the 3 germinal layers (endoderm, mesoderm, and ectoderm). Induced pluripotent stem cells (iPSC) are created by exogenously overexpressing the pluripotency markers (OCT4, SOX2, c-MYC, NANOG and KLF4) using a viral or non-viral vector, thereby inducing pluripotency to the transfected cell line.

Pluripotent stem cells are considered to be undifferentiated when they have not committed to a specific lineage. Such cells display morphological characteristics that distinguish them from differentiated cells of embryo or adult origin. Undifferentiated iPSCs are easily recognized by those skilled in the art, and typically appear in the two dimensions of a microscopic view in colonies of cells with high nuclear/cytoplasmic ratios and prominent nucleoli. Undifferentiated iPSCs express genes that may be used as markers to detect the presence of undifferentiated cells, and whose polypeptide products may be used as markers for negative selection.

The term “dendritic cell”, as used herein, means antigen-presenting cells of the mammalian immune system. Their main function is to process antigen material and present it on the cell surface to the T cells of the immune system. Dendritic cells are believed to be the only immune cells capable of activating naïve T cell responses.

The term “treating” or “treatment” refers to reducing, ameliorating reversing, alleviating, inhibiting the progress of, or preventing a disease or a medical condition such as cancer. In another aspect, the term also encompasses prophylaxis, therapy and cure. The subject or patient receiving “treatment,” or whom undergoes “treating” is any mammal in need of such treatment for cancer, including primates, and humans, and other mammals such as equines, cattle, swine and sheep; and domesticated mammals and pets.

The term “reprogramming” means inducing a pluripotent state on a differentiated somatic cell, and as used in the art.

Somatic cells of interest include, but are not limited to, fibroblasts, blood cells, urine cells, etc.

As used herein, an “adjuvant” is an immunological agent that boosts the immunological response of the recipients' immune system to target the pluripotent stem cells. The adjuvant includes those disclosed in the present application and those known in the art for boosting the immunological response of the recipients' immune system to target the pluripotent stem cells. The term “adjuvant” refers to any substance or agent that can stimulate an immune response. Some adjuvants can cause activation of a cell of the immune system. For example, an adjuvant can cause an immune cell to produce and secrete a cytokine. Examples of adjuvants that can cause activation of a cell of the immune system include, but are not limited to, the nanoemulsion formulations described herein, CG-enriched oligodeoxynucleotides (CpG), Bacillus Calmette-Guérin (BCG), activators of the STING pathway, MPL (3-O-desacyl-4′-monophosphoryl lipid A), AS04 adjuvant and mycobacteria cell wall peptidoglycans, saponins purified from the bark of the Q saponaria tree, such as QS21, poly di(carboxylatophenoxy)phosphazene (PCPP polymer; Virus Research Institute, USA); derivatives of lipopolysaccharides such as monophosphoryl lipid A (MPL; RibilmmunoChem Research, Inc., Hamilton, Mont.), muramyl dipeptide (MDP; Ribi) and threonyl-muramyl dipeptide (t-MDP; Ribi); 0M-174 (a glucosamine disaccharide related to lipid A; OM Pharma SA, Meyrin, Switzerland); cholera toxin (CT), and Leishmania elongation factor (a purified Leishmania protein; Corixa Corporation, Seattle, Wash.); or a mixture thereof. Other adjuvants known in the art may include, for example, aluminum phosphate or hydroxide salts. In some embodiments, the pluripotent stem cells are administered with one or more adjuvants. In some embodiments, the adjuvants employed are described in US2005158329; US2009010964; US2004047882; or U.S. Pat. No. 6,262,029; adjuvants for cancer vaccines are reviewed in William S. Bowen et al., 2018, Current challenges for cancer vaccine adjuvant development, Expert Review of Vaccines, 17:3, 207-215.

As used herein, the clause “an amount effective to boost (or induce) an immune response” (for example, a composition for inducing or boosting an immune response), refers to the dosage level or amount required (for example, when administered to a mammal) to stimulate, generate and/or elicit an immune response in the mammal. An effective amount can be administered in one or more administrations over different time periods, as disclosed herein (for example, via the same or different route). The application or dosage is not intended to be limited to a particular formulation or an administration route or time period.

A tumor-associated antigen (TAA) or tumor-specific antigen (TSA), as used herein, refers to known and also unknown antigens/epitopes present on cancer cells.

An optimal immune response with the cancer vaccine is to prime the host's immune system to target these TAAs and TSAs, present on pluripotent cells, and provide immunity to cancer types that express the TAAs and TSAs. Known TAAs and TSAs include, but are not limited to, EPCAM, CEACAM, TERT, WNK2 and survivin, etc.

### Methods of Vaccination:

Pluripotent stem cells as a source for the cancer vaccine can be obtained from any mammalian species, including, for example, human, primate, equine, bovine, porcine, etc. but particularly human cells. For induced pluripotent cells, multiple starting tissues or cells can be use including, without limitation, blood cells, skin cells, fibroblasts and epithelial cells.

Pluripotent stem cells are produced and grown using standard methods known in the art, preferably in feeder cell free conditions until a stable pluripotent stem cell population is formed (Sun N., Panetta NJ, Gupta DM, Wilson KD, Lee A, Jia F, Hu S, Cherry AM, Robbins RC, Longaker MT, Wu JC. Feeder-free derivation of induced pluripotent stem cells from adult human adipose stem cells. Proc Natl Acad Sci USA. 2009 Sep 15;106(37):15720-5; Jia F,

Wilson KD, Sun N, Gupta DM, Huang M, Li Z, Panetta NJ, Chen ZY, Robbins RC, Kay MA, Longaker MT, Wu JC. A nonviral minicircle vector for deriving human iPS cells Nat Methods. 2010 Mar; 7(3):197-9; K. Turksen et al. (Eds) Induced Pluripotent Stem (iPS) Cells: Methods and Protocols. 2016. Humana Press. ISBN 978-1-4939-3054-8). Said population includes>90% pure pluripotent stem cell percentage as assessed by pluripotent stem cell sorting using magnetic antibody sorting (MACS) or fluorescent antibody sorting (FACS).

In one aspect of the disclosed vaccine composition, the vaccine composition, the amount of CpG given per dose is 1 mg, 2 mg, 3 mg, 4 mg, 5 mg, 6 mg, 7 mg, 8 mg, 9 mg or 10 mg. In another aspect of the vaccine composition, the number of iPS cells given per dose is about 10 million, 25 million, 50 million, 100 million, 200 million, 400 million, 800 million or 2 billion.

In another aspect, the amount of CpG per dose may be 1 mg, that is given or administered along with a number of iPS cells that is about 10 million, 25 million, 100 million, 200 million, 400 million, 800 million or 2 billion. The cell dose (range from 1×10⁶ to 1×10⁹) used for the cancer vaccine may need to be adjusted to the mammal that the vaccine is used for. In small rodents, effectiveness of the vaccine was set at 2×10⁶ pluripotent stem cells per dose.

For clinical applications as a cancer vaccine, dosage is expressed in numbers of cells and milligrams of CpG per dose. As used herein, the term “CpG” refers to a synthetic immunomodulatory oligonucleotide with an unmethylated deoxycytidylyldeoxyguanosine dinucleotide motif (reviewed in Kayraklioglu et al. in Angela Sousa (ed.), DNA Vaccines: Methods and Protocols, Methods in Molecular Biology, vol. 2197, https://doi.org/10.1007/978-1-0716-0872-2_4, Springer Science+Business Media, LLC, part of Springer Nature 2021; Feher K. Protein Pept Sci 20:1060-1068, 2019.and Campbell JD Methods Mol. Biol. 1494:15-27, 2017; each of which is hereby incorporated by reference in its entirety). Many such CpG oligonucleotides have been described in the literature.

In one embodiment, an effective amount of CpG is used. The term “effective amount” is as defined herein and also refers to the amount of CpG needed together with iPS cells to induce an immune response to said iPS cells in a mammal. It can be appreciated that the determination of an effective amount is empirical and will depend on the species of the mammal being treated, the number of iPS cells that are co-administered and the dosing route and schedule.

In one embodiment, the CpG is selected from the group consisting of CpG 1018, CpG 7909, SD-101, CpG 10104, CpG 55.2, CpG 10101, CpG 52364, MGN1703 and DV281. In one embodiment, the CpG used is CpG 1018.

In another embodiment the amount of CpG given per dose is 1 mg, 2 mg, 3 mg, 4 mg, 5 mg, 6 mg, 7 mg, 8 mg, 9 mg or 10 mg. In a specific embodiment, the amount per dose is 3 mg. In one embodiment, the number of iPS cells given per dose is about 10 million, 25 million, 50 million, 100 million, 200 million, 400 million, 800 million or 2 billion. In a specific embodiment the number of iPS cells given per dose is 100 million.

In one embodiment, the iPS cells and the CpG are administered in a single dosage form. In one embodiment, the iPS cells and the CpG are administered separately; or are administered sequentially. In one embodiment, the iPS cells and CpG are administered in pharmaceutically acceptable solutions, which may routinely contain pharmaceutically acceptable concentrations of salt, buffering agents, preservatives, compatible carriers, adjuvants, and optionally other therapeutic ingredients.

In one embodiment, the iPS cells and CpG are administered as a parenteral injection (subcutaneous, intradermal, intravenous, parenteral, intraperitoneal, intrathecal, etc.), or mucosal (intranasal, intratracheal, inhalation, and intrarectal, intravaginal etc). An injection may be in a bolus or a continuous infusion. The iPS cells and CpG can be microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533, 1990, which is incorporated herein by reference.

In a particular embodiment, the iPS cells and the CpG are delivered as a bolus subcutaneous injection.

For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units. Multiple administration of doses at specific intervals of weeks or months apart is usual for boosting the antigen-specific responses.

In a specific embodiment, the iPS cells and CpG are delivered as a course of four weekly injections every four months.

In one variation, the method comprises in-vitro generation of the iPSC-based vaccine and vaccinating, such as subcutaneously vaccinating the recipient for several weeks, including consecutive weeks, for example, 4 consecutive weeks. In one variation, the vaccination is performed weekly for at least 2 consecutive weeks, 3 consecutive weeks, 4 consecutive weeks, 5 consecutive weeks, or at least 6 consecutive weeks. In another variation, the vaccine comprises the use of iPSC together with an adjuvant, wherein the adjuvant is an immunological agent, such as an antibody, peptide or small molecule, to boost or enhance the immune response towards the vaccine.

In one embodiment, the adjuvant is selected from the group consisting of saponin formulations, virosomes, virus like particles, non-toxic derivatives of enterobacterial lipopolysaccharide (LPS), immunostimulatory oligonucleotides (e.g. an immunostimulatory oligonucleotide containing a CpG motif), mineral containing compositions, oil-emulsions, polymers, micelle-forming adjuvants (e.g., a liposome), immunostimulating complex matrices (e.g., ISCOMATRIX), particles, squalene, phosphate, cationic liposome-DNA complexes (CL DC), DDA, DNA adjuvants, gamma-insulin, ADP-ribosylating toxins, detoxified derivatives of ADP-ribosylating toxins, Freund's complete adjuvant, Freund's incomplete adjuvant, muramyl dipeptides, monophosphoryl Lipid A (MPL), poly IC, CpG oligodeoxynucleotides (ODNs), imiquimod, QS21, adjuvant system ASO, adjuvant system AS02, adjuvant system AS03, MF59®, poly di(carboxylatophenoxy)phosphazene; derivatives of lipopolysaccharides such as monophosphoryl lipid A, muramyl dipeptide (MDP; Ribi), threonyl-muramyl dipeptide (t-MDP; Ribi); OM-174; cholera toxin (CT), and Leishmania elongation factor and aluminum or aluminum salts (e.g. alum, aluminum phosphate, aluminum hydroxide). In one variation, the adjuvant is AS101. Other suitable adjuvants include TLR agonists, NOD agonists and lipid-DNA agonist complexes.

In one embodiment, the adjuvant is admixed with the iPSC cells. In one embodiment, the adjuvant is simply injected together with the iPSC cells. In another embodiment, the adjuvant is incubated for a period of time with the iPSC cells so that the adjuvant is taken up by the iPSC cells either by nonspecific mechanisms such as pinocytosis or by receptor mediated mechanisms. In one variation, the incubation is performed for at least 1 hour, 2 hours, 6 hours, 12 hours or 24 hours. In one embodiment the iPSC cells are emulsified in an adjuvant. In one variation, the emulsifying agent used selected from the group consisting of oil-in-water, saponins, squalenes, QS21, adjuvant system ASO, adjuvant system AS02 and adjuvant system AS03. In another embodiment, the adjuvant and the iPSC cells are incorporated into a delivery system. In one variation, the delivery system is a resorbable matrix. In one variation the resorbable matrix is selected from the group consisting of a hydrogel, a collagen gel, an alginate, poly(lactic acid) (PLA), poly(lactide-co-glycolide) (PLGA) and polycaprolactone. In one embodiment the adjuvant is covalently attached to the cells. In one embodiment the adjuvant is contained in a nanoparticle. In one variation, the nanoparticle is selected from the group consisting of gold, poly(lactic acid) (PLA), poly(lactide-co-glycolide) (PLGA) and polycaprolactone nanoparticles. In one variation, the nanoparticle is simply injected together with the iPSC cells. In another variation, the adjuvant is incubated for a period of time with the iPSC cells so that the nanoparticle is taken up by the iPSC cells either by nonspecific mechanisms such as pinocytosis or by receptor mediated mechanisms. In one variation, the incubation is performed for at least 1 hour, 2 hours, 6 hours, 12 hours or 24 hours. In one variation the nanoparticle is covalently attached to the iPSCs.

In one embodiment, the pluripotent stem cells are not genetically engineered. In one embodiment, the pluripotent stem cells are genetically engineered using standard methods known in the art. Delivery of genes to pluripotent stem cells can be accomplished, for example, with viral vectors including, but not limited to, lentiviral, adenoviral, adeno-associated viral and sendai viral vectors. Delivery of genes to pluripotent stem cells can also be accomplished by delivery of DNA plasmids, DNA minicircles or RNA to the cells. This delivery method can be accomplished by electroporation, nanoparticle delivery or through the use of lipids as well as other means known in the art.

In one embodiment, the pluripotent stem cells are not genetically engineered to express one or more immune cell binding proteins (e.g., ICAM1, LFA-1, LFA-3, CD80, CD81, CD28, ICOS, 4-1BB, anti-DEC-205 antibody, anti-CLEC9A (DNGR) antibody, anti-DCIR-2 antibody, anti-DECTIN antibody, anti-ASGPR antibody, anti-mannose receptor antibody, anti-CLEC12 (DCAL-2) antibody or combination thereof). In another embodiment, the pluripotent stem cells are genetically engineered to express one or more immune cell binding proteins (e.g., ICAM1, LFA-1, LFA-3, CD80, CD81, CD28, ICOS, 4-1BB, anti-DEC-205 antibody, anti-CLEC9A (DNGR) antibody, anti-DCIR-2 antibody, anti-DECTIN antibody, anti-ASGPR antibody, anti-mannose receptor antibody, anti-CLEC12 (DCAL-2) antibody or combination thereof). In one variation, the pluripotent stem cells are genetically engineered to express CD28 and CD80.

In another embodiment, the immune cell binding proteins improve antigen cross-presentation in antigen presenting cells. The level of cross presentation in antigen presenting cells can be determined by methods known in the art including, but not limited to, measuring the expression of peptides on MHC proteins on dendritic cells with flow cytometry, measuring the level of T cells activated by cross presenting dendritic cells in vitro and measuring cytotoxic T cell activation in vivo.

In one embodiment, the pluripotent stem cells are not genetically engineered to express one or more cytokines (e.g., XCR1, CCL3, CCL4, CCL5, CCL20, CCL25 and FLT3L, or combination thereof). In another embodiment, the pluripotent stem cells are engineered to express CCL3 and FLT3L. In another variation, iPSCs are genetically engineered to secrete proteins selected from the group comprising GM-CSF, INF-alpha, INF-beta, IL-2, IL-12, IL-15 and IL-21, or a combination thereof. In another embodiment, the allogeneic vaccine expresses IL-15. In another variation, iPSCs are genetically engineered to express proteins selected from the group comprising gp96, hsp90, hsp70, CD91, calreticulin and LOX-1. In one embodiment, the iPSCs are genetically engineered to express hsp70. In another variation, the autologous or allogeneic vaccine is genetically engineered to express a cell surface protein selected from the group comprising B7, OX40, CD28, CD40L, TLR4, CD70, MHC Class I, MHC Class II and OX40L. In another embodiment, the iPSCs are engineered to express OX40. In another embodiment, the iPSCs are engineered to expressed connexin-43.

In one embodiment, the level of expression of said genetically engineered proteins is adjusted to demonstrate chemotaxis or activation of immune cells. In one variation the strength of the promotor used is adjusted to obtain the desired level of expression. In another variation the copy number of the genes being expressed is adjusted to obtain the desired level of expression. In another variation, the number of pluripotent stem cells transduced in the vaccine is adjusted to obtain the desired level of expression. Chemotaxis of immune cells can be measure by methods known in the art including, but not limited to, in vitro assays with Boyden chambers and in vivo assays looking at immune infiltrates at the site of vaccination. Activation of immune cells can be measured by methods known in the art including, but not limited to, measuring the levels of antigens on the cells using flow cytometry or immunocytochemistry, measuring the number of activated immune cells by Elispot, measuring the number of activated immune cells by cytotoxicity assays or other means known in the art.

In another variation, the autologous or allogeneic vaccine is genetically engineered to contain an inhibitory RNA. In one variation, the inhibitory RNA is selected from the group consisting of an antisense RNA, an siRNA, an shRNA, a miRNA, a lncRNA, a pri-miRNA, an antisense oligonucleotide and a pre-miRNA. In one variation, the inhibitory RNA inhibits expression of a gene selected from the group consisting of MHC Class I, MHC Class II, Beta2 microglobulin and LAMP. In one embodiment the inhibitory RNA inhibits Beta2 microglobulin. In one variation, the inhibitory RNA inhibits expression of a gene selected from the group consisting of PD-1, PDL-1, PDL-2, Nodal, cytokine signaling 1 (SOCS1), IL-10, IL-10R, TGF-β and TGF-β. In another variation, the inhibitory RNA inhibits expression of TGF-β. In one variation the inhibitory RNA is expressed at sufficiently high concentration to inhibit expression of one or more of these genes in antigen-presenting cells that take up the iPSC vaccine. Those skilled in the art can use methods other than inhibitory RNA, for example methods involving CRISPR, to inhibit the expression of particular genes in the autologous or allogeneic vaccine.

In one embodiment, an allogeneic vaccine is developed using CRISPR to delete the genes for beta 2 microglobulin and HLA-DR. The inventors have surprisingly found that the presence of these genes direct to the immune system to primarily attack the allogeneic antigens rather than be focused on the cancer-related genes of interest. Without meaning to be bound by a particular theory, the inventors have found that deleting beta 2 microglobulin and HLA-DR forces the immune system of the recipient to respond primarily to cross-presented antigens and therefore make an allogeneic product that is effective in inducing an immune response to cancer.

In another aspect, the autologous or allogeneic vaccine is administered in combination with a small molecule drug. In one embodiment, the drug is selected from the group consisting of an HDAC inhibitor, a bromodomain inhibitor, a Vps34 kinase inhibitor, a PRMT5 inhibitor, an autophagy inhibitor, an angiogenesis inhibitor, a vascular disruption agent, an activator of the STING pathway and an activator of the Toll receptor pathway. In another variation, the small molecule drug is an activator of STING plus an activator of the Toll receptor pathway.

In one variation, the HDAC inhibitor is selected from the group consisting of valproic acid, givinostat, belinostat, entinostat, mocetinostat, practinostat, chidamide, quisinostat abexinostat, vorinostat, romidepsin, panobinostat and belinostat. In another variation, the HDAC inhibitor is vorinostat.

In one variation, the BET inhibitor is selected from the group consisting of I-BET 151 (GSK1210151A), I-BET 762 (GSK525762), OTX-015, TEN-010. CPI-203 and CPI-0610. In another variation, the BET inhibitor is CPI-0610.

In one variation the Vps34 kinase inhibitor is selected from the group consisting of SB02024 and SAR405. In another variation, the PRMT5 inhibitor is GSK3326595. In one variation, the autophagy inhibitor is selected from the group consisting of 3-methyladenine, bafilomycin A1, chloroquine, hydroxychloroquine, N˜2˜-(1H-benzimidazol-6-yl)-N˜4˜-(5-cyclobutyl-1H-pyrazol-3-yl)quinazoline-2,4-diamine, MRT68921, MRT67307, SBI-0206965, ULK100, ULK101 and SB02024. In one variation, the autophagy inhibitor is SB02024.

In one variation, the angiogenesis inhibitor is selected from the group consisting of axitinib, bevacizumab, cabozantinib, everolimus, lenalidomide, lenvatinib mesylate, pazopanib, regorafenib, sorafenib, sunitinib, thalidomide, vandetanib and ramucirumab. In another variation, the angiogenesis inhibitor is sorafenib.

In one variation, the vasculature disrupting agent is selected from the list consisting of combretastatin, AVE8062, ZD6126, ABT-571, MN-029, CKD516, OXi8006, 5,6-dimethylxanthenone 4-acetic acid, combretastatin A-4 phosphate, ZD6126, Oxi4503 and the dolatastatin derivative TZT-1027.

In one variation, the STING activator is selected from the group consisting of ADU-S100, MK-1454, MK-2118, BMS-986301, SR-717, GSK3745417, SB-11285, IMSA-101, c-di-GMP, c-di-AMP and cGAMP. In one variation, the STING activator is SR-717.

In one variation, the Toll pathway activator is selected from the group consisting of diacyl lipopeptide, triacyl lipopeptide, flagellin, poly I:C, hexa-acetylated lipid A, monophosphoryl lipid A, gardiquimod, imiquimod and R848. In another variation, the Toll pathway activator is imiquimod.

In another variation, the autologous or allogeneic vaccine is co-administered with an antibody selected from the group consisting of anti-CD47, rituximab, cetuximab, daratumumab, trastuzumab, trastuzumab emtansine, pertuzumab, panitumumab, ramucirumab, necitumumab and blinatumomab. In one variation, the autologous or allogeneic vaccine is co-administered with blinatumomab.

In another variation, the autologous or allogeneic vaccine is co-administered with a small molecule drug selected from the group consisting of ibrutinib, acalabrutinib and galuniseritib. In another variation, the autologous or allogeneic vaccine is co-administered with ibrutinib.

In one variation, the small molecule drug and the iPSC cells are incorporated into a delivery system. In one variation, the delivery system is a resorbable matrix. In one variation the resorbable matrix is selected from the group consisting of a hydrogel, a collagen gel, an alginate, poly(lactic acid) (PLA), poly(lactide-co-glycolide) (PLGA) and polycaprolactone. In one embodiment the small molecule drug is contained in a nanoparticle. In one variation the nanoparticle is selected from the group consisting of gold, poly(lactic acid) (PLA), poly(lactide-co-glycolide) (PLGA) and polycaprolactone nanoparticles. In one variation, the nanoparticle is injected together with the iPSC cells. In another variation, the adjuvant is incubated for a period of time with the iPSC cells so that the nanoparticle is taken up by the iPSC cells either by nonspecific mechanisms such as pinocytosis or by receptor mediated mechanisms. In one variation, the incubation is performed for at least 1 hour, 2 hours, 6 hours, 12 hours or 24 hours. In one variation, the nanoparticle is covalently attached to the iPSCs.

In one variation, the small molecule drug is given orally, parentally or intravenously together with the iPSC vaccine. In another variation, the small molecule drug is given 4 days before the vaccine, 3 days before the vaccine, 2 days before the vaccine, 1 day before the vaccine, simultaneously with the vaccine, 1 day after the vaccine, 3 days after the vaccine, 4 days after the vaccine or any combination of days before, during and after the vaccine is administered.

In one aspect, the pluripotent stem cells are induced to undergo a specific type of cell death. The inventors have surprisingly discovered that the pluripotent stem cells do not need to be viable at the time of injection in order to elicit the desired anti-cancer immunity.

In one embodiment, iPSCs are killed in a manner to induce the expression of calreticulin on the cell surface.

In one embodiment, iPSC that are killed by in vitro administration of oxaliplatin or doxorubicin and induced to express calreticulin prior to admixture with adjuvant are particularly potent.

In one variation, the cell death-inducing agent is selected from the group comprising glutamate, sorafenib, ML162, FIN56, FIN02, erastin, sulfazine, RSL3, Ki8751, SGX-523, AZD7762, KW-2449, NVP-TAE684, AZD4547, TG-101348, bleomycin, axitinib, cytochalasin B, dasatinib, SNX-2112, Semagacestat, CHIR-99021, B02, olaparib, silmitasertib, tanespimycin, nintedanib, ML031, canertinib, SMER-3, BCL-LZH-4, SN-38, tamatinib, ML334 diastereomer, analogues, salts or derivatives thereof. In another variation, the cell death-inducing agent is bleomycin.

In one variation, the cell death-inducing agent is incubated with the iPSC cells for one hour, two hours, 6 hours, 12 hours or 24 hours. In another variation, the small molecule drug is incubated with the iPSC cells for two hours and the cells harvested and frozen. In one variation, the cell death-inducing agent is given orally, parentally or intravenously together with the iPSC vaccine. In one variation, the cell death-inducing agent is given 4 days before the vaccine, 3 days before the vaccine, 2 days before the vaccine, 1 day before the vaccine, simultaneously with the vaccine, 1 day after the vaccine, 3 days after the vaccine, 4 days after the vaccine or any combination of days before, during and after the vaccine is administered.

In another embodiment, the vaccine is comprised of dendritic cells that have been obtained from the patient to be treated, grown in tissue culture and pulsed with antigens from the pluripotent stem cells prior to being delivered back to the patient. Said dendritic cells can be pulsed with whole pluripotent stem cells, extracts from pluripotent stem cells, mRNA from pluripotent stem cells, cDNA from pluripotent stem cells or proteins or peptides from pluripotent stem cells.

In another embodiment, there is provided a method for the vaccination of a mammal with a pluripotent stem cell cancer vaccine, the method comprising: introducing the mammalian pluripotent stem cells from 1) an embryonic source, or 2) by reprogramming from a somatic cell from the recipient; and providing the recipient with pluripotent stem cells. In another aspect, the mammalian cells are undifferentiated pluripotent cells.

In another aspect of the method, the pluripotent stem cells are genetically engineered from a patient's tumor cells. Any cell type from the tumor can be used but in a stem cell population of a tumor may be used. The stem cell population from a tumor can be isolated by a number of methods known in the art.

In another embodiment, the pluripotent stem cells are genetically engineered from normal cells and fused with a patient's tumor cells. Fusion of cells can be accomplished by methods known in the art such as electrical cell fusion, polyethylene glycol cell fusion, sendai virus induced cell fusion and optically controlled thermoplasmonics.

In one embodiment, the vaccine is irradiated prior to vaccination to prevent growth of the iPSCs. In one variation, growth is prevented by exposing of the iPSCs to a cross-linker of DNA instead of radiation. In one variation, the cross-linker is selected from the group comprising nitrogen mustards, cisplatin, BCNU, psoralens and mitomycin C. The amount of cross-linking used is sufficient to prevent the iPSCs from undergoing cell division. Additional DNA cross-linkers can be used provided they inhibit cell division and do not interfere with the vaccine properties of the iPSC. In one variation of the method, the vaccine is subcutaneously injected for the duration of less than or equal to 4 weeks, such as 3 weeks, 2 weeks or about 1 week. In another variation, the vaccination is performed weekly. In another variation, the vaccination may be performed daily, several times a week such as twice or three times a week, or every two weeks, and the duration may be two, three, four, five, six, seven, or 8 weeks, or more.

A therapeutically effective dose of the vaccine can boost or enhance the in vivo immune response to a cancer by at least about 10%, at least about 20%, at least about 30%, at least about 50%, at least about 75%, at least about 90% or more, relative to the effect in the absence of administering the vaccine of the present application. Assays used to measure T-cell response include, but not limited to, delayed-type hypersensitivity testing, flow cytometry using peptide major histocompatibility complex tetramers, lymphoproliferation assay, enzyme-linked immunosorbant assay (ELISA), enzyme-linked immunospot assay (ELISpot), cytokine flow cytometry, cytotoxic T-lymphocyte (CTL) assay, CTL precursor frequency assay, T-cell proliferation assays, carboxyfluorescein diacetate succinimidyl ester assays, polyfunctional T-cell assays, measurement of cytokine mRNA by quantitative reverse transcriptase polymerase chain reaction (RT-PCR), RNAseq and limiting dilution analysis.

Analysis of tumor biopsies can be used to monitor immune responses to the cancer vaccine. Said biopsies can be assessed for infiltration of immune cells into the tumor. The immune cells can be assessed by immunocytochemistry, flow cytometry, Q-TOF, ELISpot, RNAseq, or any of the methods known in the art or described above.

Responses to the cancer vaccine can also be assessed by expression of cell surface antigens on immune cells. Increased expression of antigens such as CD69, 0×40, HLA-DR and CD154 and/or decreased expression of antigens such as CD25, PD-L1 and TIGIT can be used to measure activation of immune cells either in the tumor or in the circulation.

Other assays to evaluate immune responses include, but not limited to, gene expression profiling, protein microarrays to evaluate antibody responses to multiple antigens at one time, luciferase immunoprecipitation, phosphoflow for measuring multiple intracellular signaling molecules in the immune system at a single-cell level for lymphocyte immune monitoring, and surface plasmon resonance biosensors to monitor antibody immunity in serum.

Clinical indications of effectiveness include, but are not limited to, reduction of tumor size by imaging technologies including, but not limited to, MRI or PET scan, reduction in biomarkers including, but not limited to, PSA, AFP, CA19-9, CA-125, CEA, circulating tumor cells, circulating miRNA and clinical parameters including, but not limited, morbidity and mortality.

In one variation of the method, the vaccination causes destruction of tumor cells. These tumor cells release small molecules, peptides, proteins or genetic material into the circulation that can be detected and serve as biomarkers of vaccine activity. The biomarkers can increase in blood, plasma, serum, urine, fecal matter or other bodily fluids as the vaccine induces immune responses that kill the tumor cells. The biomarkers can also decrease with time as the tumor cells that produce them decrease in quantity.

In one embodiment, the biomarkers are detected by methods known in the art including, but not limited to, PCR, ELISA, flow cytometry or analytical chemistry.

In one embodiment a protein biomarker is selected from the group comprising lactate dehydrogenase, αl-Acid glycoprotein, fucosylated αl-acid glycoprotein, PSA, PSA-3, CEA, CA19-9, CA15-3, CA27.29, NMP-22, Calcitonin, thyroglobulin, HCG-β, Alfa-fetoprotein, HER-2, MAGEA3, NY-ESO-1, PMEL and IGFBP2. In one embodiment, the biomarker is cell-free DNA. In one embodiment, the biomarker is lncRNA or miRNA. In one embodiment, the miRNA is selected from the group comprising mi-24, mi-320a, mi-423-5q, miR-21-5p, miR-20a-5p, miR-141-3p, miR-145-5p, miR-155-5p, and miR-223-3p, miR-23a-3p, miR-27a-3p, miR-142-5p, miR-376c-3p, miR-642b-3p, miR-1202-5p, miR-1207-5p, miR-1225-5p, miR-4270-5p, miR-1825-3p and miR-4281-3p.

In another embodiment, the biomarkers are expressed in the tumor. In one variation, the biomarker is a measure of tumor mutational burden (TMB). In another variation, the biomarkers are tumor expression of one or more proteins selected from the group comprising PDL-1, PDL-2, TGFbeta, VEGF, CXCL12, CCL18, ARG1, iNOS, IL-10, IL-35 and Galectin-1.

In another variation of the method the vaccine is administered prophylactically to a patient that has a family history or genetic abnormality (including, but not limited to, mutations in BRCA1, BRCA2, APC, FAP, HNCC, TP53, P16 and PTEN) prior to the development of a cancer. In another variation of the method, the vaccine is administered prophylactically to healthy individuals prior to the development of a cancer.

In another embodiment, there is provided a thermally stable vaccine composition comprising an effective amount of mammalian pluripotent stem cells obtained from an embryonic source or obtained by reprogramming of somatic cells from a mammalian, and optionally, an adjuvant or an immunological agent to boost the immune response towards the vaccine.

In one variation, iPSC are killed prior to forming a thermally stable vaccine by in vitro administration of oxaliplatin or doxorubicin and induced to express calreticulin prior to admixture with adjuvant are particularly potent. In one variation, the cell death-inducing agent is selected from the group comprising glutamate, sorafenib, ML162, FIN56, FIN02, erastin, sulfazine, RSL3, Ki8751, SGX-523, AZD7762, KW-2449, NVP-TAE684, AZD4547, TG-101348, bleomycin A2, axitinib, cytochalasin B, dasatinib, SNX-2112, Semagacestat, CHIR-99021, B02, olaparib, silmitasertib, tanespimycin, nintedanib, ML031, canertinib, SMER-3, BCL-LZH-4, SN-38, tamatinib, ML334 diastereomer, analogues, salts or derivatives thereof.

In another aspect of the vaccine composition, the pluripotent stem cells are induced pluripotent stem cells (iPSCs). In another aspect of the vaccine composition, the mammalian pluripotent stem cells are undifferentiated pluripotent stem cells. In another aspect of the vaccine composition, the pluripotent stem cells are manufactured from the group consisting of fibroblast, keratinocytes, peripheral blood cells and renal epithelial cells. In another aspect of the vaccine composition, the pluripotent stem cells are killed with an optimal concentration and duration of exposure to oxaliplatin or doxorubicin to induce expression of calreticulin. In one variation, the cell death-inducing agent is selected from the group comprising glutamate, sorafenib, ML162, FIN56, FIN02, erastin, sulfazine, RSL3, Ki8751, SGX-523, AZD7762, KW-2449, NVP-TAE684, AZD4547, TG-101348, bleomycin A2, axitinib, cytochalasin B, dasatinib, SNX-2112, Semagacestat, CHIR-99021, B02, olaparib, silmitasertib, tanespimycin, nintedanib, ML031, canertinib, SMER-3, BCL-LZH-4, SN-38, tamatinib, ML334 diastereomer, analogues, salts or derivatives thereof.

In one variation, the application discloses a formulation for use in the treatment of cancer in a patient, comprising administration to vaccination of the patient with a vaccine, wherein the vaccine comprises an effective amount of mammalian pluripotent stem cells obtained from an embryonic source or obtained by reprogramming of somatic cells from the patient, wherein the vaccination comprising the step of administering a mammalian pluripotent stem cells to the patient in need thereof.

In one embodiment, there is provided a vaccination for use in treating cancer in a patient wherein the vaccine comprises an effective amount of mammalian pluripotent stem cells, or fragments thereof, obtained from an embryonic source or obtained by reprogramming of somatic cells from the patient, wherein the vaccination comprising the step of administering mammalian pluripotent stem cells to the patient in need thereof. In another variation, the vaccine is a thermally stable vaccine composition comprising an effective amount of mammalian pluripotent stem cells obtained from an embryonic source or obtained by reprogramming of somatic cells from a mammal, and an adjuvant or an immunological agent to boost the immune response towards the vaccine. In another aspect, the vaccine is a combination of mammalian pluripotent stem cells obtained from an embryonic source or obtained by reprogramming of somatic cells from a mammal and that have been killed with an optimal concentration and duration of exposure to oxaliplatin or doxorubicin to induce expression of calreticulin. and an adjuvant or an immunological agent to boost the immune.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.), but some experimental errors and deviations should be accounted for. Unless indicated otherwise, parts are parts by weight, molecular weight is weight average molecular weight, temperature is in degrees Centigrade (° C.), and pressure is at or near atmospheric.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

For further elaboration of general techniques useful in the practice of the methods of the present application, the practitioner can refer to standard textbooks and reviews in cell biology, tissue culture and embryology. With respect to tissue culture and ESCs, see Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, TakaRa, Thermofisher, Sigma-Aldrich and Qiagen.

### Example 1: Allogeneic iPSC Vaccine Using shRNA

In one particular aspect, the present inventors have found that autologous iPSC vaccines are more potent than allogeneic iPSC vaccines. The major difference between autologous and iPSC vaccines are the differences in major histocompatibility loci (MHC Class I and MHC Class II). MHC mismatches may provoke intense immune responses. For example, in mice, skin grafts that are matched at MHC loci are acutely rejected in a week or so whereas those that are mismatched at minor histocompatibility loci take much more time to reject and may remain intact for over a month.

In the case of the iPSC vaccine, the overwhelming immune response to MHC differences obviates the development of immune responses to the cancer-related antigens of interest. It is therefore desirable, in this case, to remove the MHC antigens in iPSC used for allogeneic vaccines.

In one method, siRNA or shRNA is used to silence expression of beta2-microglobulin. Lentiviral particles containing β-2-Microglobulin shRNA plasmid are obtained from a commercial source such as Santa Cruz Biotechnology (Santa Cruz, Calif.) and used according to the manufacturer's instructions. In brief, iPSC cells are plated in a 12-well tissue culture plate 24 hours prior to viral infection at a concentration such that they are ˜50% confluent on the day of infection (Day 2). On Day 2, iPSC medium is prepared together Polybrene® (sc-134220) at a final concentration of approximately 5 μg/ml (depending on the lot of Polybrene, with each lot tested for optimal concentration without inducing toxicity). The medium in the iPSC culture plate is removed and replaced with 1 ml of the Polybrene/media mixture per well (for 12-well plate). The lentiviral particles are thawed and added to the cells at a concentration depending on the lot of lentivirus (with each lot tested for optimal concentration of particles yielding maximal gene inhibition prior to incubation). The particles are incubated with the cells overnight then washed and fresh medium added to the plates.

In order to obtain stable expression of the shRNA, puromycin selection is used. Puromycin is added to the culture at the highest concentration that does not kill untransfected cells, usually from 2 to 10 μg/mL. The culture is continued in the presence of puromycin and puromycin-resistant clones are picked and expanded in the presence of puromycin to the desired number of cells used for vaccine.

The effectiveness of the shRNA can be assessed by PCR of beta 2 microglobulin or by FACS analysis of the cells using antibodies for HLA Class I using standard methods known in the art. Knocking out beta 2 microglobulin is sufficient to inhibit the expression of HLA Class I. Similar methods can be used to inhibit expression of HLA Class II using shRNA directed against individual Class II members such as HLA-DR.

### Example 2: Allogeneic iPSC Vaccine Using CRISPR

An alternative to using shRNA is using CRISPR technology. Two gene specific gRNAs are designed around 5′ end of the coding sequence for beta 2 microglobulin are obtained in as part of a CRISPR knockout kit from a commercial vendor such as Origene (Rockville, Md.) and used according to the manufacturer's instructions.

In brief, approximately 18-24 hours before transfection, plate ˜3×10⁵ adherent iPSC cells are plated in 2 ml culture media into each well of a 6-well plate, with the exact concentration adjusted to obtain 50-70% confluence on the following day. One (1)μg of each of the gRNA vectors is diluted in 250 μL of Opti-MEM I (Life Technologies) and vortexed gently. Then one (1)μg of the donor DNA is diluted into the same 250 μL of Opti-MEM I. Vortex gently. Six (6)μL of Turbofectin 8.0 is added to the diluted DNA (not the reverse order) and then pipetted gently to mix completely (initial titrations are conducted to see if the 3:1 ratio of Turbofectin 8.0 to DNA is optimal or if another ratio increases transfection efficiency). The mixture is then incubated for 15 minutes at room temperature after which it's drop-wise to the cells without changing the media. The plate gently rocked plate back-and-forth and side-to-side to distribute the complex evenly and the cells incubated in a 5% CO2 incubator.

48 Hours post transfection the cells are split 1:10 and grown additional 3 days; the cells are the cells again 1:10 and the process repeated in order to split the cells 2-4 times in total.

In order to select for the cells in which the CRISPR knockout was successful, puromycin selection is used. Puromycin is added to the culture at the highest concentration that does not kill untransfected cells, usually from 2 to 10m/mL. The culture is continued in the presence of puromycin and puromycin-resistant clones are picked. These clones then can be expanded as needed in the absence of puromycin.

The effectiveness of the CRISPR knockout can be assessed by PCR of beta 2 micrglobulin or by FACS analysis of the cells using antibodies for HLA Class I using standard methods known in the art. Knocking out beta 2 microglobulin is sufficient to inhibit the expression of HLA Class I. Similar methods can be used to inhibit expression of HLA Class II using gRNA vectors directed against individual Class II members such as HLA-DR.

### Example 3: Enhanced Allogeneic Vaccine

An iPS cell line is genetically engineered to not express beta 2 microglobulin or Class II HLA antigens as in Example 2. The cells are then infected with adeno-associated viral particles containing CD28 and CD80 expression cassettes (Vector BioLabs, Malvern Pa.) according to the instructions of the manufacturer. In brief, the virus-containing media is prepared by thawing the virals stock and adding the desired amount of virus to growth media to achieve the desired multiplicity of infection (MOI). This is calculated using the following equation:

AAV GC particles to be used=MOI(multiplicity of infection)* #of cells to be infected

The optimal MOI is determined using green fluorescent protein (GFP) expressing AAV from the same vendor with MOIs ranging from 2,000 to 500,000 and looking for optimal GFP expression by flow cytometry.

To infect with the AAV viruses containing the CD28 and the CD80 expression cassettes the cell culture media is removed and AAV-containing media is added to the cell culture and incubated for 6 hours.

Levels of CD28 and CD80 on the iPS cells are monitored using flow cytometry.

### Example 4: Fusion of Cancer Cells and iPS

The iPSC vaccine presents a large number of embryonic antigens in common with cancer cells. However, cancer cells may also contain antigens caused by somatic mutations that are unique to each individual cancer. In order to broaden the antigen display of iPSC the inventors have discovered that fusion of an individual's cancer cells with iPSCs allows for the additional expression of these antigens.

Tissue from a tumor biopsy is dissociated by means known in the art using collagenase. Briefly, tumor tissue from surgical resection is placed in tissue culture medium minced with a scalpel to obtain˜1-3 mm³ pieces. The minced tissue is transferred into a 15 mL or 50 mL conical tube, depending on the amount of tissue, and the tube centrifuged at 100×g at room temperature for 5 min. A solution containing collagenase II is added to achieve 1 mg/mL final concentration and a solution of DNase I is added to achieve a final concentration of 100 Kunitz/mL (the final concentrations are adjusted depending on the lot of enzyme).

The mixture is placed in a flask which is placed on a rocking platform and allowed to rock at room temperature until the tissue is visibly dissociated.

The resulting cell suspension is placed in 15 mL or 50 mL conical tube and washed three times with serum-free tissue culture medium. The cells are resuspended in serum-free tissue culture medium and counted. A cell suspension of iPS cells in serum-free tissue culture medium is added at a ratio of 2 cancer cells for every iPS cell. The cells are centrifuged for 5 minutes at 100×g at room temperature in serum-free tissue culture medium. The supernatant is removed and the cell pellet gently resuspended by tapping the bottom of the tube. Fusion is performed by slowly adding 1 ml 50% PEG 1500 pre-warmed to 37° C. to the pellet using a 1 ml pipette, over a period of 1 min, continually mixing the cells by gentle shaking for an additional 2 minute. Three (3) ml of medium pre-warmed to 37° C. is slowly added over a period of 3 min while continuously stirring the cells by gentle shaking. Ten (10) ml of medium pre-warmed to 37° C. is then slowly added, the cells incubated for another 10 minutes and then centrifuged at 100×g for 5 minutes. The cells are then washed one more time to remove residual PEG and prepared for injection or frozen using standard cryopreservation techniques.

All references, including publications, books, patent applications, and patents, cited herein are hereby incorporated by reference to the same extent as if each reference were individually and specifically indicated to be incorporated by reference and were set forth in its entirety herein.

### REFERENCES

1. Jia, F. et al. A nonviral minicircle vector for deriving human iPS cells. Nat Methods 7,197-199 (2010).
2. Warren, L. et al. Highly efficient reprogramming to pluripotency and directed differentiation of human cells with synthetic modified mRNA. Cell Stem Cell 7, 618-630 (2010).
3. Chou, B. K. et al. Efficient human iPS cell derivation by a non-integrating plasmid from blood cells with unique epigenetic and gene expression signatures. Cell Res 21, 518-529 (2011).
4. Okita, K. et al. A more efficient method to generate integration-free human iPS cells. Nat Methods 8, 409-412 (2011).
5. Mandal, P. K. & Rossi, D. J. Reprogramming human fibroblasts to pluripotency using modified mRNA. Nat Protoc 8, 568-582 (2013).

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

What is claimed:

1. A mammalian autologous vaccine or allogeneic vaccine comprising an effective amount of a mammalian induced pluripotent stem cells (iPSCs) obtained by reprogramming of somatic cells from a patient:
wherein the autologous vaccine or the allogeneic vaccine expresses a gene selected from the group consisting of ASTE1, BIRC5, CDCA1, CDKN2A, DEPDC1, EGFR, ERBB2, FOXM1, GPC3, HJURP, HSPA8, HSP90B1, IDH1, IDO1, IGF2BP3, IMP3, KIF20A, KIF20B, MELK, MGATS, NUF2, PMEL, RAS, TAF1B, TOMM34, TTK, TP53, VEGFR1 and VEGFR2;
wherein the autologous vaccine or the allogeneic vaccine optionally may contain an adjuvant; and
wherein the autologous vaccine or the allogeneic vaccine induces an immune response from the patient for the treatment of cancer.

2. The vaccine of claim 1 where the gene or genes selected are determined by the type of cancer in the patient selected from the group consisting of:

| | |
|---|---|
| Astrocytoma | IDH1; |
| Bladder | DEPDC1 KIF20B; |
| Breast | BIRC5 CDCA1 DEPDC1 ERBB2 KIF20A |
| | KIF20B; |
| Cervical | FOXM1 HJURP MELK; |
| Colorectal | ASTE1 IGF2BP3 TAF1B TOMM34 VEGFR1 |
| | VEGFR2; |
| Esophageal | CDCA1 IGF2BP3 IMP3 TTK; |
| Gastric | ERRB2; |
| Glioblastoma | EGFR HSP90B1; |
| Head and Neck | CDCA1 CDKN2A IMP3; |
| Liver | GPC3 HSPA8; |
| Melanoma | HSP90B1 MGAT5 PMEL; |
| NSCLC | ERBB2 HSP90B1 IDO1 IMP3 NUF2 TTK TP53 |
| | VEGFR1 VEGFR2; |
| Ovarian | BIRC5 ERBB2 FOXM1 HJURP MELK VEGFR1 |
| | VEGFR2; |
| Pancreatic | ERBB2 HSPA8 KIF20A RAS TP53 VEGFR1 |
| | VEGFR2; and |
| Prostate | BIRC5 ERBB2. |

3. A mammalian autologous vaccine or allogeneic vaccine comprising an effective amount of a mammalian induced pluripotent stem cells (iPSCs) obtained by reprogramming of somatic cells from a patient:
wherein the autologous vaccine or the allogeneic vaccine is genetically engineered to contain an inhibitory RNA not involved in the reprograming process; and
wherein the autologous vaccine or the allogeneic vaccine induces an immune response from the patient for the treatment of cancer.

4. The vaccine of claim 3 wherein the inhibitory RNA is selected from the group consisting of antisense RNA, siRNA, shRNA, miRNA, lncRNA, pri-miRNA, an antisense oligonucleotide and a pre-miRNA.

5. The vaccine of claim 4 wherein the inhibitory RNA inhibits the expression of a gene selected from the group consisting of MHC Class I, MHC Class II, Beta2 microglobulin and LAMP.

6. The vaccine of claim 5 wherein the inhibitory RNA inhibits the expression of a gene selected from the list comprising PD-1, PDL-1, PDL-2, Nodal, cytokine signaling 1 (SOCS1), IL-10, IL-10R, TGF-β and TGF-βR.

7. A mammalian autologous vaccine or allogeneic vaccine comprising an effective amount of a mammalian induced pluripotent stem cells (iPSCs) obtained by reprogramming of somatic cells from a patient:
wherein the autologous vaccine or the allogeneic vaccine is genetically engineered to express a gene not involved in the reprograming process; and
wherein the autologous vaccine or the allogeneic vaccine induces an immune response from the patient for the treatment of cancer.

8. The vaccine of claim 7 wherein the gene is selected from the group consisting of ICAM1, LFA-1, LFA-3, CD80, CD81, CD28, ICOS, 4-1BB, anti-DEC-205 antibody, anti-CLEC9A (DNGR) antibody, anti-DCIR-2 antibody, anti-DECTIN antibody, anti-ASGPR antibody, anti-mannose receptor antibody and anti-CLEC12 (DCAL-2) antibody.

9. The vaccine of claim 7 wherein the gene is selected from the group consisting of XCR1, CCL3, CCL4, CCL5, CCL20, CCL25 and FLT3L.

10. The vaccine of claim 7 wherein the gene is selected from the group consisting of GM-CSF, INF-alpha, INF-beta, IL-2, IL-12, IL-15 and IL-21.9.

11. The vaccine of claim 7 wherein the gene is selected from the group consisting of gp96, hsp90, hsp70, CD91, calreticulin and LOX-1.

12. The vaccine of claim 7 wherein the gene is selected from the group consisting of B7, OX40, CD28, CD40L, TLR4, CD70, MHC Class I, MHC Class II and OX40L.

13. The mammalian autologous vaccine or allogeneic vaccine claim 1, wherein the mammalian induced pluripotent stem cells are non-viable and express a protein on the cell surface selected from the group consisting of calreticulin, Hsp70 and HSP90.

14. The mammalian autologous vaccine or allogeneic vaccine of claim 13, wherein the mammalian induced pluripotent stem cells are killed by in vitro administration of a chemotherapeutic agent.

15. The mammalian autologous vaccine or allogeneic vaccine of claim 14, wherein the chemotherapeutic agent is selected from the group consisting of oxaliplatin and doxorubicin.

16. The mammalian autologous vaccine or allogeneic vaccine claim 1, wherein the vaccine further comprises of dendritic cells, where the dendritic cells are obtained from the patient.

17. The mammalian autologous vaccine or allogeneic vaccine of claim 16, wherein the dendritic cells are pulsed with iPS cells reprogrammed from adult cells obtained from the patient.

18. The mammalian autologous vaccine or allogeneic vaccine of claim 16 wherein the dendritic cells are grown in tissue culture and pulsed with antigens from the induced pluripotent stem cells prior to being delivered back to the patient.

19. The mammalian autologous vaccine or allogeneic vaccine of claim 18, wherein the dendritic cells are selected from the group of dendritic cells pulsed with whole pluripotent stem cells, extracts from pluripotent stem cells, mRNA from pluripotent stem cells, cDNA from pluripotent stem cells or proteins or peptides from pluripotent stem cell.

20. The mammalian autologous vaccine or allogeneic vaccine of claim 1, wherein the cancer is selected from the group consisting of leukemia, multiple myeloma, Hodgkin's lymphoma, non-Hodgkin's lymphoma, lymphoma, myeloproliferative disorders, squamous cell cancer, adenocarcinoma, sarcoma, neuroendocrine carcinoma, bladder cancer, skin cancer, brain and spinal cord cancers, head and neck cancer, thyroid, bone cancer, breast cancer, cervical cancer, colorectal cancer, endometrial cancer, gastrointestinal cancers, (hypo)laryngeal cancer, esophageal cancer, liver cancer, lung cancer, pancreatic cancer, prostate cancer, eye cancer, renal cell cancer, kidney, hepatic, ovarian cancer, gastric cancer, testicular cancer, thyroid and thymus cancer.

21. The mammalian autologous vaccine or allogeneic vaccine composition of claim 20, wherein the adjuvant is CpG and the amount of CpG per dose is 1 mg, 2 mg, 3 mg, 4 mg, 5 mg, 6 mg, 7 mg, 8 mg, 9 mg or 10 mg.

22. The mammalian autologous vaccine or allogeneic vaccine composition of claim 21, wherein the number of iPS cells per dose is 10 million, 25 million, 50 million, 100 million, 200 million, 400 million, 800 million or 2 billion.
